(19) United States
(12) Patent Application Publication
(10) Pub. No.: US 2016/0131880 A1
(43) Pub. Date: May 12, 2016

(54) MEDICAL OBSERVATION APPARATUS, MEDICAL VIDEO MICROSCOPE APPARATUS, AND MEDICAL VIDEO MICROSCOPE SYSTEM

(71) Applicant: Sony Olympus Medical Solutions Inc., Tokyo (JP)

(72) Inventors: Yoshiyuki KAMATA, Tokyo (JP); Shigeru TAMURA, Tokyo (JP); Kenji HIROSE, Tokyo (JP)

(73) Assignee: Sony Olympus Medical Solutions Inc., Tokyo (JP)

(21) Appl. No.: 14/879,265

(22) Filed: Oct. 9, 2015

(30) Foreign Application Priority Data
Nov. 12, 2014 (JP) 2014-229736

(51) Int. Cl. G02B 21/00; H04N 7/18; G02B 7/00

(52) U.S. Cl. CPC G02B 21/0012; G02B 7/001; H04N 7/183; A61B 19/5223; A61B 2019/264; A61B 2019/265; A61B 2019/266

(57) ABSTRACT

There is provided a medical observation apparatus including: a holding unit including a transmission mechanism including a plurality of joint units placed in positions different from each other and configured to rotate in conjunction with each other around a rotation axis in the same direction. Brakes are placed for at least two joint units of the plurality of joint units.

### CROSS REFERENCE TO RELATED APPLICATIONS

This application claims the benefit of Japanese Priority Patent Application JP 2014-229736 filed Nov. 12, 2014, the entire contents of which are incorporated herein by reference.

### BACKGROUND

The present disclosure relates to a medical observation apparatus, a medical video microscope apparatus, and a medical video microscope system.

These days, in surgical operations, a method of performing an operation while observing a surgical site with an apparatus for observation (hereinafter, occasionally referred to as an observation unit) that makes it possible to observe the surgical site with magnification is used. At this time, in order to move and fix the position and posture of the observation unit with high accuracy, a medical observation apparatus in which the observation unit is held by an arm unit is used.

Here, in the medical observation apparatus, there is a case where, when the observation unit is moved and its position is fixed, the observation unit vibrates due to the inertial force. Since such vibration causes shaking of the visual field in observation under the microscope, the working cannot be performed until the shaking has subsided; and this has been a cause of an increase in the psychological burden on the operator and a decrease in the manipulability during the operation.

Hence, for example in JP 2000-107200A, a technology is disclosed in which, in a medical observation apparatus in which a brake is provided for each joint unit that forms a rotation axis corresponding to the degree of freedom of an arm unit, the time of vibration attenuation is shortened by driving these brakes with a time difference.

On the other hand, in the medical observation apparatus, in order to ensure the operator's working space etc., there is a demand to make smaller the formation around the observation unit provided at the tip of the arm unit. However, in the medical observation apparatus described in JP 2000-107200A, since a brake is mounted also on the joint unit around the observation unit, it is difficult to downsize the formation around the observation unit.

Here, for example in JP 2014-76204A, a configuration is disclosed in which, in a medical observation apparatus including an arm unit including a parallelogram link mechanism, a brake for fixing the movement of the parallelogram link mechanism is placed on the root side of the parallelogram link mechanism (the opposite side to the tip side where the observation unit is provided). According to the configuration, there is a possibility that the formation of the tip side of the parallelogram link mechanism, that is, the formation around the observation unit can be downsized because the brake provided for the parallelogram link mechanism is placed on the root side.

### SUMMARY

However, in the technology described in JP 2014-76204A, although the formation around the observation unit can be downsized, the distance from the observation unit to the brake that fixes the movement of the parallelogram link mechanism is relatively long. Consequently, there is a possibility that the vibration of the observation unit cannot be sufficiently suppressed due to bending etc. occurring on the arms included in the parallelogram link mechanism. Thus, in the medical observation apparatuses described in JP 2000-107200A and JP 2014-76204A, it has been difficult to achieve both the downsizing of the formation around the observation unit and the suppression of the vibration of the observation unit.

Thus, in an embodiment of the present disclosure, a novel improved medical observation apparatus, a novel improved medical video microscope apparatus, and a novel improved medical video microscope system that make it possible to suppress the vibration of the observation unit more while making the formation around the observation unit smaller are proposed.

According to an embodiment of the present disclosure, there is provided a medical observation apparatus including: a holding unit including a transmission mechanism including a plurality of joint units placed in positions different from each other and configured to rotate in conjunction with each other around a rotation axis in the same direction. Brakes are placed for at least two joint units of the plurality of joint units.

According to an embodiment of the present disclosure, there is provided a medical video microscope apparatus including: a holding unit including a transmission mechanism including a plurality of joint units placed in positions different from each other and configured to rotate in conjunction with each other around a rotation axis in the same direction; and a microscope unit provided at a tip of the holding unit and including an imaging unit configured to output image information of a surgical site imaged. Brakes are placed for at least two joint units of the plurality of joint units in the holding unit.

According to an embodiment of the present disclosure, there is provided a medical video microscope system including: a video microscope apparatus configured to acquire image information of a surgical site; and a display device configured to display an image based on the image information. The video microscope apparatus includes a holding unit including a transmission mechanism including a plurality of joint units placed in positions different from each other and configured to rotate in conjunction with each other around a rotation axis in the same direction, and a microscope unit provided at a tip of the holding unit and including an imaging unit configured to output the image information imaged. Brakes are placed for at least two joint units of the plurality of joint units in the holding unit of the video microscope apparatus.

According to an embodiment of the present disclosure, brakes are placed for at least two of the joint units included in the transmission mechanism included in the holding unit (an arm unit). According to the configuration, the vibration of the observation unit can be suppressed favorably by the brake provided at a point relatively near to the observation unit. Furthermore, the function of stopping the rotation of the rotation axis in the transmission mechanism can be assigned mainly to the brake provided at a point relatively far from the observation unit; therefore, the brake provided at a point relatively near to the observation unit can be configured to be a smaller-sized brake with a relatively small fixing force of which the main objective is the vibration damping of the observation unit. Therefore, both the downsizing of the formation around the observation unit and the suppression of the vibration of the observation unit can be achieved.

As described above, according to an embodiment of the present disclosure, it becomes possible to suppress the vibration of the observation unit more while making the formation around the observation unit smaller. The effects mentioned above are not necessarily limitative ones, and any effect illustrated in the present specification or other effects that can be grasped from the present specification may be exhibited, along with the effects mentioned above or instead of the effects mentioned above.

### BRIEF DESCRIPTION OF THE DRAWINGS

FIG. 1 is an illustration diagram for describing demands on a medical observation apparatus mounted with a digital microscope;

FIG. 2 is a view showing a configuration example of a medical observation apparatus according to the embodiment; and

FIG. 3 is a schematic view in which the medical observation apparatus shown in FIG. 2 is simplified and the configuration around an O4 axis and around an O5 axis is mainly shown.

### DETAILED DESCRIPTION OF THE EMBODIMENT(S)

Hereinafter, (a) preferred embodiment(s) of the present disclosure will be described in detail with reference to the appended drawings. In this specification and the appended drawings, structural elements that have substantially the same function and structure are denoted with the same reference numerals, and repeated explanation of these structural elements is omitted.

The description is given in the following order:

1. Investigation on a common medical observation apparatus
2. Configuration of the medical observation apparatus according to the embodiment

2-1. Overall configuration

2-2. Configuration of the parallelogram link mechanism

2-3. Arrangement of brakes in the parallelogram link mechanism

3. Modification example

3-1. Modification example in which the configuration of the holding unit is different

3-2. Modification example in which the microscope unit is formed of a microscope lens body including an eyepiece

3-3. Modification example in which the configuration of the transmission mechanism is different

### 4. Supplementation

(1. Investigation on a Common Medical Observation Apparatus)

Before a preferred embodiment of the present disclosure is described, to make the present disclosure clearer, the results of the investigation on a common medical observation apparatus by the present inventors are described, and the background with which the present inventors have reached the present disclosure is described.

These days, in surgical operations, a method of performing an operation while observing a surgical site with an apparatus for observation (hereinafter, occasionally referred to as an observation unit) that makes it possible to observe the surgical site with magnification is used. The observation unit corresponds to, for example, a microscope lens body, an endoscope, or the like. In an operation in which the object is a very small region, such as in a neurosurgical operation, in order to bring the region of the object into the visual field surely, it is desired to enable the position and posture of the observation unit to be moved and fixed with high accuracy. Hence, a medical observation apparatus in which the observation unit is held by an arm unit is widely used.

As a configuration example of common medical observation apparatuses, the medical observation apparatus described in JP 2000-107200A mentioned above may be given. In JP 2000-107200A, a medical observation apparatus is disclosed in which, in order to fix the position of a microscope lens body that is the observation unit, a brake is provided for each joint unit that forms a rotation axis corresponding to the degree of freedom of an arm unit. The observation unit is moved by releasing these brakes, and the position of the observation unit is fixed by driving these brakes.

Here, in the medical observation apparatus, the observation unit may vibrate when the observation unit is moved and its position is fixed. The residual vibration at the time of braking greatly influences the operator's manipulability. Hence, it is desired for the medical observation apparatus to suppress the vibration of the observation unit occurring at the time of fixing after movement in a shorter time. The difficulty of vibration attenuation greatly depends on “the length of the arm unit (arm length),” “the weight of the observation unit,” and “the rigidity of the arm unit.” When a relatively heavy observation unit is provided at the tip of a long arm unit with a low rigidity, it is more difficult for the vibration to subside.

On the other hand, in the medical observation apparatus, there is a demand to make smaller the formation around the observation unit provided at the tip of the arm unit. In view of the fact that the position of the observation unit is fixed relatively near to the surgical site in order to observe the surgical site, the above demand is in order to avoid a hindrance by the observation unit to the working of the operator who is performing various treatments on the surgical site. The downsizing of the formation around the observation unit is effective in order also to suppress the vibration of the observation unit as mentioned above. In the case of a medical observation apparatus in which a counterweight is provided, also the counterweight can be reduced in weight in accordance with the downsizing of the formation around the observation unit; therefore, the formation of the entire arm unit can be downsized and there is also an advantage that costs can be reduced.

From the viewpoint of suppressing vibration, in JP 2000-107200A mentioned above, a technology is disclosed in which the time of vibration attenuation is shortened by driving the brake provided at each joint unit with a time difference. However, in the technology described in JP 2000-107200A, since a brake is placed also for the joint unit around the observation unit, it is difficult to downsize the formation around the observation unit, and the technology is not preferable from the viewpoint of ensuring a working space like the above. If the size of the formation around the observation unit is increased, also the size of the counterweight may be increased, and also the size of the formation of the entire arm unit may be increased.

In contrast, in JP 2014-76204A mentioned above, a medical observation apparatus is disclosed in which an arm unit including a parallelogram link mechanism is provided and a brake for fixing the movement of the parallelogram link mechanism is placed on the root side of the parallelogram link mechanism (the opposite side to the tip side where the observation unit is provided). According to the configuration, there is a possibility that the formation of the tip side of the parallelogram link mechanism, that is, the formation around the observation unit can be downsized because the brake provided for the parallelogram link mechanism is placed on the root side.

However, in the technology of JP 2014-76204A, the distance from the observation unit to the brake that fixes the movement of the parallelogram link mechanism is relatively long as compared to the case where the brake is provided on the tip side of the parallelogram link mechanism. Consequently, upon driving the brake, when bending or the like has occurred on a member (e.g. the arms included in the parallelogram link mechanism) provided between the observation unit and the brake, there is a possibility that the vibration of the observation unit cannot be sufficiently suppressed due to the bending.

Thus, in existing medical observation apparatuses like those described in JP 2000-107200A and JP 2014-76204A, it has been difficult to achieve both the downsizing of the neighborhood of the observation unit and the suppression of the vibration of the observation unit.

Here, as a medical observation apparatus including a microscope unit as the observation unit (a medical microscope apparatus), a microscope apparatus in which the microscope unit is provided with an eyepiece and with which an operation is performed while the operator looks in the eyepiece to directly observe the surgical site, as described in JP 2000-107200A, is widely used. On the other hand, these days, a video microscope apparatus in which the microscope unit is provided with an imaging unit and an image photographed by the imaging unit is displayed on a monitor is coming into use, with the progress of image processing technology and the achievement of high pixels (fineness) of the imaged image (see e.g. JP 2002-272760A etc.)

When the video microscope apparatus is used, the operator does not look in the eyepiece, but performs the operation while observing the image of the surgical site shown on the monitor. Hence, it is not preferable that an object that obstructs the operator's visual field exist between the operator and the monitor. Thus, in the video microscope apparatus, the demand for downsizing the neighborhood of the microscope unit is greater than in the microscope apparatus in which the microscope unit is provided with an eyepiece.

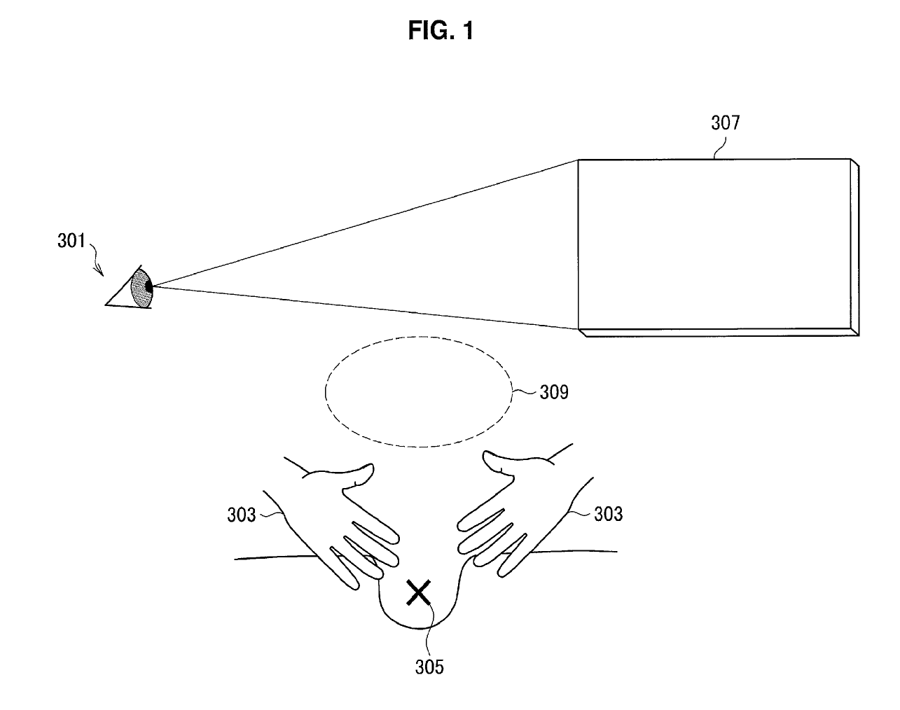

This issue will now be described with reference to FIG. 1. FIG. 1 is an illustration diagram for describing demands on the video microscope apparatus.

In FIG. 1, the positional relationships among an operator's eye 301, the operator's hands 303, a surgical site 305, and a monitor 307 on which an image photographed by a video microscope apparatus is displayed are schematically illustrated. The surgical site 305 is also the focus position of the microscope unit of the video microscope apparatus. As shown in FIG. 1, during an operation, the operator's gaze is directed to the monitor 307. During the operation, the operator's hands 303, not-illustrated surgical instruments manipulated by the operator, etc. exist near the surgical site 305 for working. Therefore, during the operation, the space that neither obstructs the operator's visual field nor interferes with the operator's hands 303, the surgical instruments, etc. is limited to a relatively small region 309 schematically illustrated in the drawing. Thus, for the video microscope apparatus, it is desired for the formation of the microscope unit and its neighborhood to fall within the region 309.

In the video microscope apparatus, by its electronic zoom function, it becomes possible to magnify the surgical site even to a region that has been difficult to observe with a microscope apparatus configured to directly observe the surgical site via an existing eyepiece. However, when the magnification is increased, the minute vibrations of the microscope unit and the arm unit greatly influence the shaking of the imaged image. The microscope unit and the arm unit may vibrate minutely due to an event where the hand is taken off the microscope unit after movement, the shaking of the neighborhood, etc.; but when it takes a long time for the vibration to attenuate, it is desired to stand by until the imaged image has come to a standstill every time the microscope unit is moved and the hand is taken off, and the operator's stress and troublesomeness of manipulation are greater than in the case of the microscope apparatus configured to directly observe the surgical site via an eyepiece.

Thus, in the video microscope observation apparatus, since it is desired for the formation around the microscope unit to fall within a relatively small space like the region 309 shown in FIG. 1, even more downsizing of the formation around the microscope unit is desired as compared to the microscope apparatus configured to directly observe the surgical site via an eyepiece. Furthermore, although the surgical site can be observed in more detail using the electronic zoom function, the influence of the vibration of the microscope unit on the shaking of the visual field is greater; thus, even more vibration damping is desired.

Here, as described above, for the sake of vibration damping, it is preferable that the arm length be set smaller, the weight of the formation around the microscope unit be set smaller, and the rigidity of the arm unit be set larger. However, to ensure the operator's working space and visual field like those described with reference to FIG. 1, it is desired for the arm unit to be placed so as to detour around the working space and the visual field; consequently, it is desired to make the arm unit still longer and thinner. If it is attempted to make the rigidity of the arm unit larger, the size of the arm unit is increased; thus, this is not preferable from the viewpoint of ensuring the operator's working space and visual field like those described above. Therefore, it is important to make the formation around the microscope unit smaller in order not only to suppress the vibration but also to ensure the operator's working space and visual field.

Hereinabove, the results of the investigation by the present inventors on a common medical observation apparatus are described. As described above, in a common existing medical observation apparatus, it has been difficult to achieve both the downsizing of the formation around the observation unit and the suppression of the vibration of the observation unit. Furthermore, in the video microscope apparatus, the demands for downsizing the formation around the observation unit (i.e. the microscope unit) and suppressing the vibration of the microscope unit are greater.

Based on the investigation results mentioned above, the present inventors made extensive research on a technology that makes it possible to suppress the vibration of the observation unit more while making the formation around the observation unit smaller, and consequently have reached the present disclosure described below. According to the embodiment, both the downsizing of the formation around the observation unit and the suppression of the vibration of the observation unit can be achieved; thus, high manipulability can be achieved even when a configuration for a video microscope is used, not to mention when a microscope unit configured to directly observe the surgical site via an eyepiece is used as the microscope unit. A preferred embodiment of the present disclosure that the present inventors have reached will now be described.

(2. Configuration of the Medical Observation Apparatus According to the Embodiment)

The configuration of a medical observation apparatus according to an embodiment of the present disclosure will now be described. In the following, a user that performs various manipulations on the medical observation apparatus according to the embodiment is written as an operator for the sake of convenience. However, the writing does not limit the user that uses the medical observation apparatus, and various manipulations on the medical observation apparatus may be performed by any user, such as any other medical staff member.

In the following, the case where the observation unit provided in the medical observation apparatus according to the embodiment is a microscope unit is described as an example. However, the embodiment is not limited to the example, and the observation unit may be formed of any other device for observing the surgical site, such as an endoscope.

(2-1. Overall Configuration)

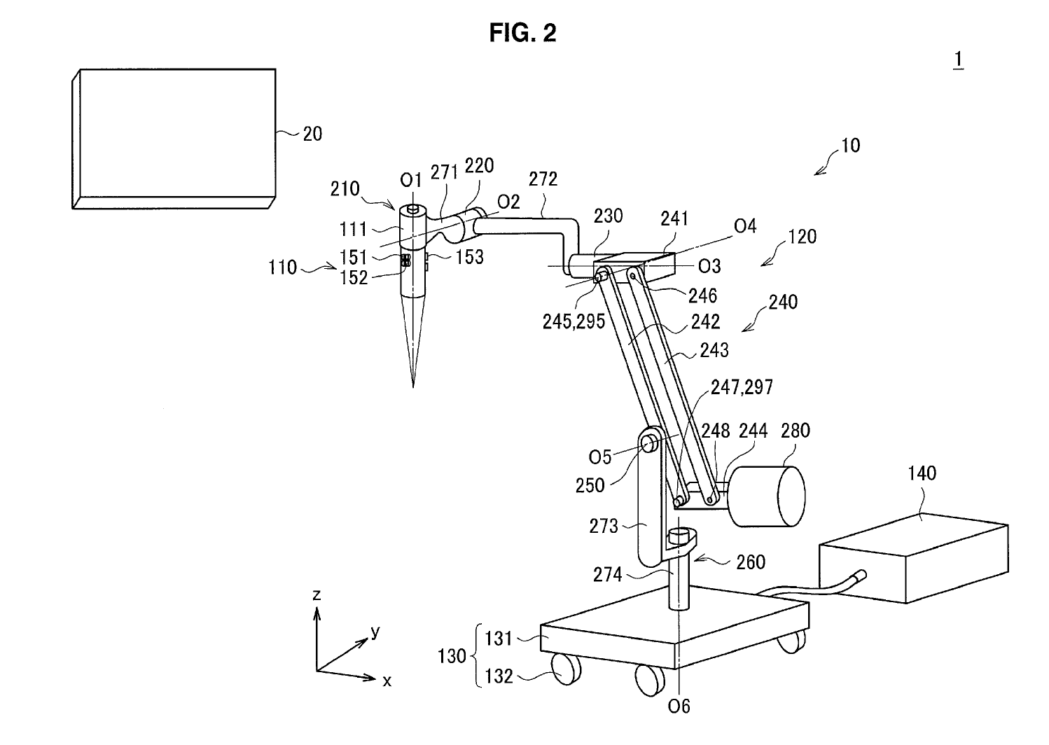

The overall configuration of the medical observation apparatus according to the embodiment will now be described with reference to FIG. 2. FIG. 2 is a view showing a configuration example of the medical observation apparatus according to the embodiment.

Referring to FIG. 2, a medical observation apparatus 10 according to the embodiment includes a microscope unit 110 for observing a surgical site of a patient with magnification, a holding unit 120 (an arm unit 120) that holds the microscope unit 110, a base unit 130 to which one end of the holding unit 120 is connected and which supports the microscope unit 110 and the holding unit 120, and a control device 140 that controls the operation of the medical observation apparatus 10. In the present specification, the medical observation apparatus 10 in which the microscope unit 110 is provided as the observation unit may be referred to as a medical microscope apparatus 10.

(Base Unit 130)

The base unit 130 supports the microscope unit 110 and the holding unit 120. The base unit 130 includes a base 131 having a plate-like shape and a plurality of casters 132 provided on the lower surface of the base 131. One end of the holding unit 120 is connected to the upper surface of the base 131, and the microscope unit 110 is connected to the other end (tip) of the holding unit 120 extended from the base 131. The medical observation apparatus 10 is in contact with the floor surface via the casters 132, and is configured to be movable on the floor surface by means of the casters 132.

In the following description, the direction vertical to the floor surface on which the medical observation apparatus 10 is placed is defined as the z-axis direction. The z-axis direction may be referred to as the vertical direction or the perpendicular direction. Two directions orthogonal to the z-axis direction and to each other are defined individually as the x-axis direction and the y-axis direction. A direction parallel to the x-y plane may be referred to as the horizontal direction.

(Microscope Unit 110)

The microscope unit 110 is formed of a microscope lens body for observing a surgical site of a patient with magnification. In the example shown in FIG. 2, the microscope unit 110 has a configuration corresponding to a video microscope, and is formed of an optical system of an objective lens, a zoom lens, etc. housed in a lens barrel and an imaging unit 111 that photographs an image of an object (i.e. a surgical site) using light that has passed through the optical system. The imaging unit 111 is formed of, for example, a video camera device, and the image information of the surgical site is acquired by the imaging unit 111. In the illustrated example, the optical axis direction of the microscope unit 110 substantially coincides with the z-axis direction. The medical observation apparatus 10 including the microscope unit 110 in which an imaging unit is provided and which can output image information in this way may be referred to as a medical video microscope apparatus 10 in the present specification.

As the microscope unit 110, configurations corresponding to various known video microscopes may be used, and a detailed description thereof is omitted here. For example, various known imaging elements such as a charge coupled device (CCD) sensor and a complementary metal-oxide-semiconductor (CMOS) sensor may be used as the imaging element of the imaging unit 111. The imaging unit 111 may be mounted with various functions that are provided in common camera devices, such as an optical zoom function. The imaging unit 111 may be configured as what is called a stereo camera including a pair of imaging elements. Also for the optical system of the microscope unit 110, various known configurations may be used. Furthermore, the microscope unit 110 may be mounted with various functions that are generally provided in the microscope units of video microscope apparatuses, such as an auto focus (AF) function and an optical zoom function.

The image information acquired by the microscope unit 110 is transmitted to the control device 140, and various image processings, such as gamma correction and the adjustment of white balance, are performed in the control device 140. In the control device 140, an image processing such as magnification and inter-pixel correction according to the electronic zoom function may be further performed. The image information that has undergone image processing is transmitted to a display device 20 provided in an operation room, and the image of the surgical site is displayed on the display device 20. As the display device 20, various known display devices, such as a cathode ray tube (CRT) display device, a liquid crystal display device, a plasma display device, and an electro-luminescence (EL) display device, may be used. The communication between the control device 140 and the display device 20 may be conducted by various known wired or wireless systems.

The image photographed by the microscope unit 110 may be magnified at a desired magnification by the optical zoom function and/or the electronic zoom function and displayed on the display device 20. The operator observes the surgical site and performs various treatments on the surgical site while referring to the imaged image that is photographed by the microscope unit 110 and displayed on the display device 20 with magnification as appropriate. Thus, in the embodiment, the medical observation apparatus 10 and the display device 20 constitute a medical observation system 1 (a medical video microscope system 1).

The microscope unit 110 may be provided with a processing circuit for performing the image processing mentioned above, and the image processing mentioned above may not be performed by the control device 140 but may be performed by the processing circuit of the microscope unit 110. In this case, the image information after image processing is performed as appropriate in the processing circuit mounted in the microscope unit 110 may be transmitted from the microscope unit 110 to the display device provided in the operation room. In this case, the communication between the microscope unit 110 and the display device 20 may be conducted by various known wired or wireless systems.

The microscope unit 110 is provided with various switches for controlling the operation of the microscope unit 110. For example, the microscope unit 110 is provided with a zoom switch 151 (a zoom SW 151) and a focus switch 152 (a focus SW 152) for adjusting the photographing conditions of the microscope unit 110, and an operating mode change switch 153 (an operating mode change SW 153) for changing the operating mode of the holding unit 120.

The operator can adjust the magnification and the focal distance of the microscope unit 110 by manipulating the zoom SW 151 and the focus SW 152. The operator can switch the operating mode of the holding unit 120 to either of a fixing mode and a free mode by manipulating the operating mode change SW 153.

Here, the fixing mode is an operating mode in which the rotation at each rotation axis provided in the holding unit 120 is regulated by a brake and thereby the position and posture of the microscope unit 110 are fixed. The free mode is an operating mode in which the brake is released and thereby the rotation at each rotation axis provided in the holding unit 120 is possible freely, and the position and posture of the microscope unit 110 can be adjusted by direct manipulation based on the manual operation by the operator. Here, the direct manipulation refers to a manipulation in which the operator grasps a grasping unit, for example with the hand, and directly moves the microscope unit 110.

For example, while the operator holds down the operating mode change SW 153, the operating mode of the holding unit 120 is the free mode; and while the operator keeps the hand off the operating mode change SW 153, the operating mode of the holding unit 120 is the fixing mode. The vibration of the microscope unit 110 described in (1. Investigation on a common medical observation apparatus) above is what may occur when the holding unit 120 is moved in the free mode and then the operating mode of the holding unit 120 is switched from the free mode to the fixing mode.

These switches may not necessarily be provided for the microscope unit 110. In the embodiment, it may be sufficient that a mechanism for receiving operating inputs which has functions equivalent to these switches be provided in the medical observation apparatus 10, and the specific configuration of the mechanism is not limited. For example, these switches may be provided in other portions of the medical observation apparatus 10. Furthermore, for example, an input device such as a remote control may be used so that orders corresponding to these switches are inputted to the medical observation apparatus 10 remotely.

Although illustration is omitted in FIG. 2 to avoid complicating the drawing, a grasping unit that is grasped by the operator may be provided in a partial region of the microscope unit 110. During the free mode, the operator can move the microscope unit 110 manually by grasping the grasping unit with the hand. Since the operator performs the manipulation of moving the microscope unit 110 while holding down the operating mode change SW 153 in a state of grasping the grasping unit, the arrangement positions of the grasping unit and the operating mode change SW 153 are preferably determined in view of the mutual relative positional relationship and the operator's manipulability.

Although the case where the microscope unit 110 has a configuration corresponding to a video microscope is described in the above, the embodiment is not limited to the example. In the embodiment, the microscope unit 110 may be formed of a microscope lens body provided with an eyepiece. The operator performs various treatments on the surgical site while looking in the eyepiece and directly observing the image of the surgical site that is magnified at an appropriate magnification by the optical system provided in the microscope unit 110.

(Control Device 140)

The control device 140 is formed of a processor, such as a central processing unit (CPU) or a digital signal processor (DSP), a microcomputer mounted with these processors, or the like, and controls the operation of the medical observation apparatus 10 by executing arithmetic processing in accordance with a prescribed program. For example, the control device 140 has a function of switching the operating mode of the holding unit 120 described above by controlling the driving of the brake provided at each joint unit of the holding unit 120 in accordance with the manipulation input by the operator via the operating mode change SW 153 mentioned above. Furthermore, for example, the control device 140 has a function of adjusting the magnification and the focal distance of the microscope unit 110 in accordance with the manipulation input by the operator via the zoom SW 151 and the focus SW 152 mentioned above. Furthermore, the control device 140 has functions of performing various image processings on the image information photographed by the microscope unit 110 and transmitting the image information after processing to the display device 20 provided in the operation room.

Although in the illustrated example the control device 140 is provided as a configuration separated from the microscope unit 110 and the arm unit 120 and is connected to the microscope unit 110 and the arm unit 120 via a cable or the like, the embodiment is not limited to the example. For example, a processor, a microcomputer, or the like that realizes similar functions to the control device 140 may be incorporated in the microscope unit 110; thereby, the control device 140 and the microscope unit 110 may be formed integrally.

(Holding Unit 120)

The holding unit 120 holds the microscope unit 110, moves the microscope unit 110 three-dimensionally, and fixes the position and posture of the microscope unit 110 after movement. In the illustrated example, the holding unit 120 is configured as a balance arm having six degrees of freedom. However, the embodiment is not to the example, and the holding unit 120 may be configured so as to have any other different number of degrees of freedom. By configuring the holding unit 120 as a balance arm to make a configuration in which the moments of the microscope unit 110 and the holding unit 120 are balanced as a whole, the microscope unit 110 can be moved by a smaller external force, and the operator's manipulability can be improved more.

In the holding unit 120, six rotation axes corresponding to six degrees of freedom are provided. In the following, the members constituting the rotation axis are collectively referred to as a rotation axis unit for convenience of description. The rotation axis unit may be formed of, for example, a bearing, a shaft inserted through the bearing in a rotationally movable manner, a brake that regulates the rotation on the rotation axis, etc. Also a parallelogram link mechanism 240 described later may be regarded as a rotation axis unit.

The holding unit 120 is composed of rotation axis units 210, 220, 230, 240, 250, and 260 corresponding to rotation axes (hereinafter, abbreviated as rotation axis units 210 to 260), arms 271, 272, 273, and 274 that make connections between the rotation axis units 210 to 260, and a counterweight 280 for balancing the moments of the microscope unit 110 and the holding unit 120 as a whole. However, the rotation axis unit 240 is formed of a parallelogram link mechanism 240. In the following, a description is given by marking the rotation axes with the names of an O1 axis to an O6 axis for the sake of convenience. The rotation axis nearest to the microscope unit 110 is the O1 axis, and the rotation axis nearest to the base unit 130 is the O6 axis.

The rotation axis unit 210 is provided so that the microscope unit 110 can rotationally move around, as the rotation axis direction, a rotation axis (the O1 axis) substantially coinciding with the optical axis of the microscope unit 110. The microscope unit 110 rotationally moves around the O1 axis by means of the rotation axis unit 210; thereby, the direction of the imaged image by the microscope unit 110 is adjusted.

One end of the arm 271 extending in a direction substantially perpendicular to the O1 axis is connected to the rotation axis unit 210. The other end of the arm 271 is provided with the rotation axis unit 220 configured so that the arm 271 can rotationally move around, as the rotation axis direction (the O2 axis direction), a direction substantially parallel to the extending direction of the arm 271. The O2 axis is placed substantially perpendicularly to the O1 axis, and is provided as a rotation axis substantially parallel to the y-axis in the example shown in FIG. 2. The microscope unit 110 and the arm 271 rotationally move around the O2 axis as the rotation axis by means of the rotation axis unit 220; thereby, the position in the x-axis direction of the microscope unit 110 is adjusted.

One end of the arm 272 extending in a direction substantially perpendicular to both the O1 axis and the O2 axis is connected to the rotation axis unit 220. The other end of the arm 272 is bent in a substantially L-shaped configuration; and the position corresponding to the short side of the bend is provided with the rotation axis unit 230 configured so that the arm 272 can rotationally move around, as the rotation axis direction (the O3 axis direction), the extending direction of the arm 272 (the long side direction of the L shape). The O3 axis is placed substantially perpendicularly to the O1 axis and the O2 axis, and is provided as a rotation axis substantially parallel to the x-axis in the example shown in FIG. 2. The microscope unit 110, the arm 271, and the arm 272 rotationally move around the O3 axis as the rotation axis by means of the rotation axis unit 230; thereby, the position in the y-axis direction of the microscope unit 110 is adjusted.

One end of the upper side of the parallelogram link mechanism 240 is connected to an end of the rotation axis unit 230 on the side to which the arm 272 is not connected. The parallelogram link mechanism 240 is formed of four arms (arms 241, 242, 243, and 244) arranged in a parallelogram configuration and four joint units (joint units 245, 246, 247, and 248) provided individually in positions corresponding to substantial vertices of the parallelogram.

Specifically, one end of the arm 241 extending in a direction substantially parallel to the O3 axis is connected to the rotation axis unit 230. In other words, the arm 272 and the arm 241 are arranged as arms extending in substantially identical directions. One end of the arm 241 is provided with the joint unit 245, and the other end is provided with the joint unit 246. One ends of the arms 242 and 243 are connected to the joint units 245 and 246, respectively, in a rotationally movable manner around rotation axes substantially parallel to each other (the O4 axis) which are inserted through the joint units 245 and 246, respectively. The other ends of the arms 242 and 243 are provided with the joint units 247 and 248, respectively. The arm 244 is joined to the joint units 247 and 248 so as to be rotationally movable around the rotation axis (the O4 axis) inserted through the joint units 247 and 248 and be substantially parallel to the arm 241.

Thus, the four joint units included in the parallelogram link mechanism 240 have rotation axes in directions substantially parallel and substantially identical to each other (the O4 axis), and move around the O4 axis in conjunction with each other. In the example shown in FIG. 2, the O4 axis is provided as a rotation axis substantially parallel to the y-axis. In other words, the parallelogram link mechanism 240 is configured so as to include a plurality of joint units that are placed in positions different from each other and rotate around a rotation axis in the same direction in conjunction with each other, and behaves as a transmission mechanism that transmits the movement at one end to the other end.

By the parallelogram link mechanism 240 being provided, the movement of the formation on the tip side from the parallelogram link mechanism 240 (i.e. the microscope unit 110, the rotation axis units 210, 220, and 230, and the arms 271 and 272) is transmitted to the root side of the parallelogram link mechanism 240 (the side relatively near to the base unit 130). In the following description, when simply the tip side or the root side is written, it refers to the tip side or the root side, respectively, with the parallelogram link mechanism 240 as a reference unless otherwise specified. A more detailed configuration of the parallelogram link mechanism 240 is described later in (2-2. Configuration of the parallelogram link mechanism) below.

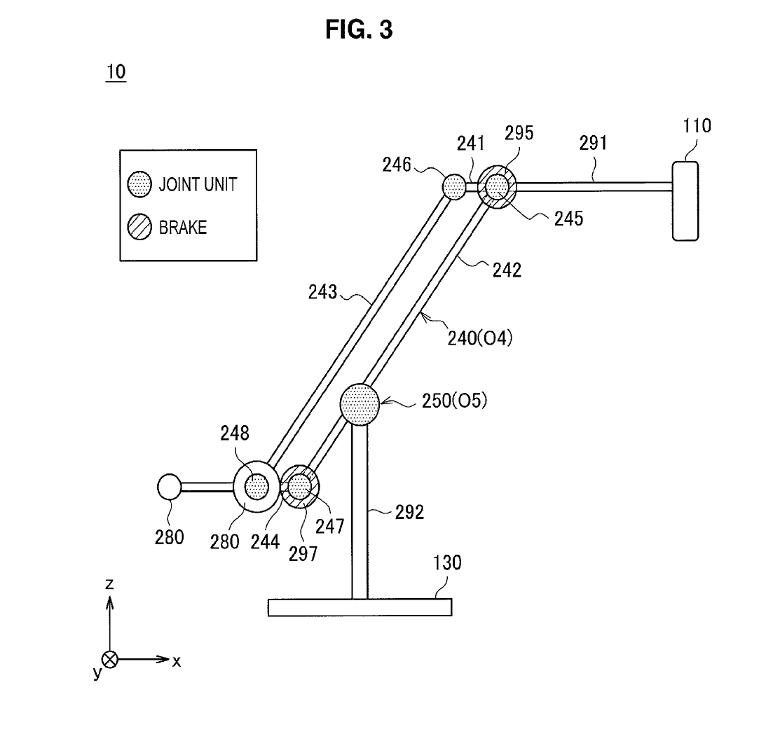

A portion of the arm 242 apart at a prescribed distance from the end where the joint unit 247 is provided is provided with the rotation axis unit 250 that supports the parallelogram link mechanism 240 in a rotationally movable manner around, as the rotation axis direction (the O5 axis direction), a direction perpendicular to the extending direction of the arm 242 (see also FIG. 3 described later). The O5 axis is a rotation axis substantially parallel to the O4 axis, and is provided as a rotation axis substantially parallel to the y-axis in the example shown in FIG. 2. One end of the arm 273 provided to extend in the z-axis direction is connected to the rotation axis unit 250; and the microscope unit 110, the arm 271, the arm 272, and the parallelogram link mechanism 240 are configured to be rotationally movable with respect to the arm 273 around the O5 axis as the rotation axis via the rotation axis unit 250.

The arm 273 has a substantially L-shaped configuration, and the opposite side to the side where the rotation axis unit 250 is provided is bent so as to be substantially parallel to the floor surface. A surface of the arm 273 substantially parallel to the floor surface is provided with the rotation axis unit 260 that allows the arm 273 to rotationally move around a rotation axis (the O6 axis) orthogonal to the O5 axis. In the example shown in FIG. 2, the O6 axis is provided as a rotation axis substantially parallel to the z-axis. The other end of the arm 274 that forms the rotation axis unit 260 is connected to the upper surface of the base 131 of the base unit 130. The microscope unit 110, the arm 271, the arm 272, the parallelogram link mechanism 240, and the arm 273 rotationally move with respect to the base unit 130 around the O6 axis as the rotation axis via the rotation axis unit 260.

The arm 244 of the parallelogram link mechanism 240 is formed to be longer than the arm 241, and one end of the arm 244 located diagonally opposite to a portion of the parallelogram link mechanism 240 to which the rotation axis unit 230 is connected is extended to the outside of the parallelogram link mechanism 240. The end of the extended arm 244 is provided with the counterweight 280 (a counterbalance 280). The mass and arrangement position of the counterweight 280 are adjusted so that the rotation moment occurring around the O4 axis and the rotation moment occurring around the O5 axis resulting from the mass of the formation placed on the tip side from the parallelogram link mechanism 240 (i.e. the microscope unit 110, the rotation axis units 210, 220, and 230, and the arms 271 and 272) can be canceled.

The arrangement position of the rotation axis unit 250 corresponding to the O5 axis is adjusted so that the center of gravity of the formation placed on the tip side from the rotation axis unit 250 (i.e. the microscope unit 110, the rotation axis units 210, 220, and 230, the arms 271 and 272, and the parallelogram link mechanism 240) is located on the O5 axis. The arrangement position of the rotation axis unit 260 corresponding to the O6 axis is adjusted so that the center of gravity of the formation placed on the tip side from the rotation axis unit 260 (i.e. the microscope unit 110, the rotation axis units 210, 220, 230, and 250, the arms 271, 272, and 273, and the parallelogram link mechanism 240) is located on the O6 axis. By the counterweight 280 and the rotation axis units 250 and 260 being thus configured, when the operator attempts to directly move the microscope unit 110 manually, the microscope unit 110 can be moved by a smaller force just like non-gravity. Therefore, the user's manipulability can be improved.

The counterweight 280 may be attachable and detachable. For example, in the case where several kinds of counterweights 280 having masses different from each other are prepared and the formation placed on the tip side from the parallelogram link mechanism 240 is changed, a counterweight 280 that can cancel the rotation moment may be selected as appropriate in accordance with the change.

The rotation axis units 210 to 260 of the holding unit 120 are provided with brakes that regulate the rotations at the rotation axis units 210 to 260, respectively. The driving of these brakes is controlled by the control device 140. These brakes may be released all at once by the control from the control device 140; thereby, the operating mode of the holding unit 120 transitions to the free mode. Similarly, these brakes may be driven all at once by the control from the control device 140; thereby, the operating mode of the holding unit 120 transitions to the fixing mode.

Here, since the four joint units of the parallelogram link mechanism 240 rotate in conjunction with each other, to stop the rotation around the O4 axis, it may be sufficient that at least one of the four joint units be provided with a brake. In the embodiment, brakes are placed individually for at least two of the four joint units included in the parallelogram link mechanism 240. In the illustrated example, brakes 295 and 297 are provided for the joint units 245 and 247 of the parallelogram link mechanism 240, respectively. By thus arranging brakes in the parallelogram link mechanism 240, the vibration of the microscope unit 110 can be suppressed more when the microscope unit 110 is moved and fixed. The reason why the vibration of the microscope unit 110 is suppressed by the configuration according to the embodiment is described in detail in (2-3. Arrangement of brakes in the parallelogram link mechanism) below.

As the brakes provided for the rotation axis units 210 to 260, various brakes used for common balance arms may be used, and the specific mechanism thereof is not limited. For example, these brakes may be a brake that is mechanically driven, or may be an electromagnetic brake that is driven electrically.

Hereinabove, the overall configuration of the medical observation apparatus 10 and the configuration of the medical observation system 1 according to the embodiment are described with reference to FIG. 2.

(2-2. Configuration of the Parallelogram Link Mechanism)

The configuration of the parallelogram link mechanism shown in FIG. 2 will now be described in more detail with reference to FIG. 3. FIG. 3 is a schematic view in which the medical observation apparatus shown in FIG. 2 is simplified and the configuration around the O4 axis and the O5 axis is mainly shown.

Referring to FIG. 3, the tip side of the parallelogram link mechanism 240 is provided with an arm 291 and the microscope unit 110. The arm 291 is what is illustrated as one member on behalf of the rotation axis units 210, 220, and 230 and the arms 271 and 272 shown in FIG. 2 for the sake of simplicity.

As described above, the parallelogram link mechanism 240 is formed of the four arms 241, 242, 243, and 244 arranged in a parallelogram configuration and the four joint units 245, 246, 247, and 248 provided individually in positions corresponding to substantial vertices of the parallelogram. The four joint units 245, 246, 247, and 248 have a bearing structure that axially supports a member in a rotationally movable manner around, as the rotation axis direction, directions substantially parallel and identical to each other (the O4 axis direction).

The arm 241 corresponding to the upper side of the parallelogram extends substantially parallel to the x-axis direction (the O3 axis direction shown in FIG. 2), and one end of the arm 241 is connected to the rotation axis unit 230 shown in FIG. 2. One end of the arm 241 is provided with the joint unit 245, and the other end is provided with the joint unit 246. One ends of the arms 242 and 243 are connected to the joint units 245 and 246, respectively. The arms 242 and 243 are connected to the arm 241 via the joint units 245 and 246, respectively, in a rotationally movable manner around the O4 axis. The arms 242 and 243 are formed to be longer than the arms 241 and 244 corresponding to the upper side and the lower side of the parallelogram, respectively, and form the long sides of the parallelogram.

The other ends of the arms 242 and 243 are provided with the joint units 247 and 248, respectively. The arm 244 is joined to the joint units 247 and 248 so as to be substantially parallel to the arm 241. The arm 244 is connected to the arms 242 and 243 via the joint units 247 and 248 in a rotationally movable manner around the O4 axis.

By employing such a structure, when the vertical position of the microscope unit 110 changes, the four joint units 245, 246, 247, and 248 of the parallelogram link mechanism 240 rotate in conjunction with each other, and the vertical movement of the microscope unit 110 is transmitted to the root side of the parallelogram link mechanism 240. Thus, the parallelogram link mechanism 240 has a function as a transmission mechanism that transmits the movement of the member provided on the tip side to the root side.

A portion of the arm 242 apart at a prescribed distance from the end where the joint unit 247 is provided is provided with the rotation axis unit 250 that supports the parallelogram link mechanism 240 in a rotationally movable manner around the O5 axis as the rotation axis. The O5 axis is substantially parallel to the O4 axis. The rotation axis unit 250 and the base unit 130 are connected by an arm 292 extending in the vertical direction. The parallelogram link mechanism 240 is connected to the arm 292 via the rotation axis unit 250 in a rotationally movable manner with respect to the arm 292 around the O5 axis as the rotation axis direction. The arm 292 is what is illustrated as one member on behalf of the rotation axis unit 260 and the arms 273 and 274 shown in FIG. 2 for the sake of simplicity.

One end of the arm 244 is extended to the outside of the parallelogram link mechanism 240, and the extended portion, that is, the root portion of the parallelogram link mechanism 240 is provided with the counterweight 280. The arrangement position, shape, weight, etc. of the counterweight 280 are adjusted so as to cancel the rotation moment occurring in the parallelogram link mechanism 240 upon the vertical movement of the microscope unit 110.

(2-3. Arrangement of Brakes in the Parallelogram Link Mechanism)

Here, in order to stop the vertical movement of the microscope unit 110 in the fixing mode, it is desired to stop the rotation around the O4 axis in the parallelogram link mechanism 240. Since the joint units 245, 246, 247, and 248 of the parallelogram link mechanism 240 rotate in conjunction with each other as mentioned above, to stop the rotation, it may be sufficient that at least one of the joint units 245, 246, 247, and 248 be provided with a brake.

Here, the arrangement of brakes for a parallelogram link mechanism in a common medical observation apparatus is described with reference to FIG. 3. In a common medical observation apparatus including a parallelogram link mechanism similarly to the configuration shown in FIG. 3, in many cases a brake is provided in a position corresponding to the joint unit 245 or the joint unit 247 shown in FIG. 3.

When a brake is provided in a position corresponding to the joint unit 245 (hereinafter, referred to as a common brake arrangement 1), the formation of the tip side of the parallelogram link mechanism, that is, the formation around the microscope unit is increased in size due to the brake. As described with reference to FIG. 1, the size increase of the formation around the microscope unit is not preferable from the viewpoint of ensuring the operator's working space and visual field. Furthermore, the size increase of the formation around the microscope unit leads to a size increase of the counterweight, and may therefore lead to a size increase of the formation of the entire arm unit or an increase in cost.

On the other hand, when a brake is provided in a position corresponding to the joint unit 247 (hereinafter, referred to as a common brake arrangement 2), the formation of the tip side of the parallelogram link mechanism, that is, the formation around the microscope unit can be downsized as compared to the common brake arrangement 1. For example, the common brake arrangement 2 is applied to the medical observation apparatus described in JP 2014-76204A mentioned above. However, in the common brake arrangement 2, the distance from the microscope unit to the brake that fixes the movement of the parallelogram link mechanism is longer than in the common brake arrangement 1. Therefore, the vibration of the microscope unit may not be sufficiently suppressed due to bending of a member provided between the microscope unit and the brake, such as bending of the arms included in the parallelogram link mechanism. The occurrence of bending can be suppressed by using an arm with a relatively large rigidity as the arms included in the parallelogram link mechanism; but when an arm with a large rigidity is used, the size of the arm is increased and also the size of the counterweight is increased.

Thus, in the embodiment, as shown in FIG. 3, the brakes 295 and 297 are placed for both of the joint unit 245 and the joint unit 247, respectively. When the operating mode of the holding unit 120 has transitioned to the fixing mode, the brakes 295 and 297 are driven substantially simultaneously in synchronization with the brakes provided for the other rotation axis units 210, 220, 230, 250, and 260, and fix the rotation around the O4 axis. According to the embodiment, since also the joint unit 245 relatively near to the microscope unit 110 is provided with the brake 245 unlike the common brake arrangement 2, the vibration of the microscope unit 110 can be suppressed more favorably.

Here, since the main objective of the brake 295 provided for the joint unit 245 relatively near to the microscope unit 110 is to suppress the vibration of the microscope unit 110, the brake 295 does not need to have a very large fixing force. Therefore, as the brake 295, a brake smaller in size than the brake used in the common brake arrangement 1 described above can be used. Hence, even when the joint unit 245 is provided with the brake 295, the formation around the microscope unit 110 can be downsized as compared to the common brake arrangement 1.

On the other hand, the main objective of the brake 297 provided for the joint unit 247 relatively far from the microscope unit 110 is to stop the movement of the parallelogram link mechanism 240, and the brake 297 preferably has a relatively large fixing force in order also to secure the fixing force (braking force) that stops the rotation around the O4 axis. Even when the formation of the brake 297 is relatively large, the possibility of obstructing the operator's working space or visual field like around the microscope unit 110 is low; therefore, this poses little problem. Thus, in the embodiment, the brakes 295 and 297 may be configured favorably such that the fixing force of the brake 295 provided at a point relatively near to the microscope unit 110 is smaller than the fixing force of the brake 297 provided at a point relatively far from the microscope unit 110.

The arrangement of the brakes 295 and 297 shown in FIG. 3 is only an example. In the embodiment, it may be sufficient that the brakes 295 and 297 be placed for at least two of the plurality of joint units 245, 246, 247, and 248 included in the parallelogram link mechanism 240, and the arrangement positions of the brakes and the number of brakes arranged are not limited to the example. However, the brakes 295 and 297 are preferably provided at points relatively near to and relatively far from the microscope unit 110, respectively. Thus, the brakes 295 and 297 are preferably placed for a joint unit located on the upper side (either of the joint units 245 and 246) and for a joint unit located on the lower side (either of the joint units 247 and 248), respectively, which are provided across the arms 242 and 243 corresponding to the long sides of the parallelogram link mechanism 240.

For example, the brake 295 provided at a point relatively near to the microscope unit 110 may be provided for the joint unit 246 instead of for the joint unit 245. Furthermore, for example, the brake 297 provided at a point relatively far from the microscope unit 110 may be provided for the joint unit 248 instead of for the joint unit 247. Alternatively, brakes may be placed individually for any three of the joint units 245, 246, 247, and 248. In this case, the joint units 245 and 246 located on the upper side may be provided with a small-sized brake with a relatively small fixing force, and the joint units 247 and 248 located on the lower side may be provided with a brake with a relatively large fixing force.

When there is a difference between the rigidities of the arm 242 and the arm 243 corresponding to the long sides of the parallelogram link mechanism 240, it is preferable that the brakes 295 and 297 be placed for the joint units located at both ends of the arm with the larger rigidity. For example, when the arm 242 is larger in rigidity than the arm 243, as shown in FIG. 3, the brakes 295 and 297 may be placed for the joint units 245 and 247 at both ends of the arm 242, respectively. By providing the brakes 295 and 297 for the arm with the larger rigidity on which deformation such as bending is less likely to occur during brake driving, the vibration of the microscope unit 110 during brake operation can be suppressed more.

Hereinabove, the configuration of the parallelogram link mechanism 240 of the medical observation apparatus 10 according to the embodiment is described in detail with reference to FIG. 3. As described above, according to the embodiment, the brake 295 mainly for vibration damping is provided at a point relatively near to the microscope unit 110, and the brake 297 mainly for stopping the rotation around the O4 axis is provided at a point relatively far from the microscope unit 110. As the brake 295 for vibration damping, a smaller-sized brake with a relatively small fixing force may be used. Therefore, in the medical observation apparatus 10 according to the embodiment, both the downsizing of the formation around the observation unit and the suppression of the vibration of the observation unit can be achieved.

(3. Modification Example)

Some modification examples in the embodiment described above will now be described.

(3-1. Modification Example in which the Configuration of the Holding Unit is Different)

Although the case where the holding unit 120 has six degrees of freedom and one parallelogram link mechanism 240 is provided is described in the above embodiment, the embodiment is not limited to the example. In the embodiment, the degree of freedom of the holding unit 120 is not limited, and also the positions and number of parallelogram link mechanisms 240 provided in the holding unit 120 are not limited.

For example, although the rotation axis unit 240 corresponding to the O4 axis is formed of the parallelogram link mechanism 240 in the configuration example shown in FIG. 2 and FIG. 3, a rotation axis unit corresponding to any other rotation axis (e.g. the rotation axis unit 220 corresponding to the O2 axis etc.) may be formed of a parallelogram link mechanism. Similarly to the parallelogram link mechanism 240 shown in FIG. 3, at least two brakes may be provided for the parallelogram link mechanism that forms the rotation axis unit corresponding to the other rotation axis.

Furthermore, an arrangement of brakes similar to that of the embodiment described above may be applied to a holding unit including a plurality of parallelogram link mechanisms like, for example, the medical observation apparatus described in JP 2014-76204A mentioned above. In this case, the brake arrangement according to the embodiment may be applied to each of the plurality of parallelogram link mechanisms, or the brake arrangement according to the embodiment may be applied to only one of the plurality of parallelogram link mechanisms.

Here, in the case where the brake arrangement according to the embodiment is applied to only one of the plurality of parallelogram link mechanisms, as the parallelogram link mechanism to which the brake arrangement according to the embodiment is applied, a parallelogram link mechanism connecting a portion of the holding unit which extends mainly in the vertical direction and controls the position in the vertical plane of the microscope unit (what is called a vertical arm unit) and a portion of the holding unit which extends mainly in the horizontal direction and controls the position in the horizontal plane of the microscope unit (what is called a horizontal arm unit) may be preferably selected. This is because it is presumed that such a parallelogram link mechanism connecting the vertical arm unit and the horizontal arm unit is provided at a substantially middle point of the holding unit between the tip side and the root side and the formation on the tip side from the parallelogram link mechanism is a portion that is more likely to influence the vibration of the microscope unit and may constitute an obstacle to the operator's working space and visual field (that is, a portion for which downsizing is desired).

In the case where a plurality of parallelogram link mechanisms exist, when the brake arrangement according to the embodiment is applied to a parallelogram link mechanism provided in a position relatively near to the microscope unit, the formation on the tip side from the parallelogram link mechanism is small in quantity and also small in weight; therefore, the shaking of the formation on the tip side may not pose a problem originally, and the effects by the embodiment cannot be obtained so much. In the case where the brake arrangement according to the embodiment is applied to a parallelogram link mechanism provided in a position relatively far from the microscope unit, the parallelogram link mechanism is located relatively on the root side of the holding unit originally; therefore, even when the formation around the upper side is downsized, this does not contribute much to ensuring the operator's working space and visual field like those described with reference to FIG. 1. Therefore, as the parallelogram link mechanism to which the brake arrangement according to the embodiment is applied, a parallelogram link mechanism that is provided in a position at such a distance from the root side as to constitute an obstacle to the operator's working space and visual field and at such a distance from the tip side that the vibration of the microscope unit constitutes a hindrance to working, like a parallelogram link mechanism connecting the vertical arm unit and the horizontal arm unit, may be preferably selected.

(3-2. Modification Example in which the Microscope Unit is Formed of a Microscope Lens Body Including an Eyepiece)

Although the case where the microscope unit 110 has a configuration for a video microscope is described in the above embodiment, the embodiment is not limited to the example. In the embodiment, the microscope unit 110 may be formed of a microscope lens body that includes an eyepiece and enables the operator to directly observe the surgical site via the eyepiece.

Even when the microscope unit 110 is formed of a microscope lens body including an eyepiece, by applying the brake arrangement according to the embodiment to the parallelogram link mechanism 240 of the holding unit 120, the vibration of the microscope unit 110 after movement can be suppressed, and the shaking of the operator's visual field can be subdued rapidly; thus, the manipulability during the operation can be improved. Furthermore, since the formation around the microscope unit 110 placed near the surgical site during the operation can be downsized, also the operator's working space can be ensured.

However, as described in (1. Investigation on a common medical observation apparatuses) above, when a configuration for a video microscope is used as the microscope unit 110, the operator performs the operation while observing the image of the surgical site shown on the display device 20; therefore, even more downsizing is desired for the formation around the microscope unit 110 in order also to ensure the operator's visual field. By the electronic zoom function that may be mounted in the microscope unit 110, the surgical site can be magnified even to a region that has been difficult to see in the case where the operator directly observes the surgical site via the eyepiece; but on the other hand, the influence of the vibration of the microscope unit 110 on the shaking of the visual field is greater, and vibration damping with sill higher accuracy is desired.

By applying the configuration of the holding unit 120 according to the embodiment to the case where a configuration for a video microscope for which even more downsizing and vibration damping are desired as mentioned above is used as the microscope unit 110, not to mention the case where the microscope unit 110 is formed of a microscope lens body including an eyepiece, the effects of the embodiment can be enjoyed more.

(3-3. Modification Example in which the Configuration of the Transmission Mechanism is Different)

Although the case where the parallelogram link mechanism 240 is provided as the transmission mechanism in the holding unit 120 is described in the above embodiment, the embodiment is not limited to the example. In the embodiment, the transmission mechanism may be any other configuration.

In the embodiment, it may be sufficient that the transmission mechanism be a mechanism that is configured so as to include a plurality of joint units that are placed in positions different from each other and rotate in conjunction with each other around a rotation axis in the same direction, and has a function of transmitting the movement at one end to the other end; and the specific configuration is not limited. By brakes being placed for at least two of the plurality of joint units of the transmission mechanism, similar effects to the embodiment described above can be obtained.

For example, the transmission mechanism may be a belt mechanism. The belt mechanism is formed of, for example, a plurality of pulleys and a belt provided to stretch over the pulleys. In the belt mechanism, the plurality of pulleys are arranged so as to have rotation axes in directions substantially parallel to each other, and the plurality of pulleys rotate in conjunction with each other via the belt. The holding unit according to the embodiment may be formed also by the following: a portion corresponding to any one rotation axis of the holding unit is formed of a belt mechanism, and brakes are provided for at least two of the plurality of pulleys included in the belt mechanism.

(4. Supplementation)

It should be understood by those skilled in the art that various modifications, combinations, sub-combinations and alterations may occur depending on design requirements and other factors insofar as they are within the scope of the appended claims or the equivalents thereof.

In addition, the effects described in the present specification are merely illustrative and demonstrative, and not limitative. In other words, the technology according to the present disclosure can exhibit other effects that are evident to those skilled in the art along with or instead of the effects based on the present specification.

For example, although in the above embodiment the arm unit 120 of the medical observation apparatus 10 is provided with an observation unit for observing a surgical site, the present technology is not limited to the example. For example, the arm unit 120 may be equipped with any other surgical instrument used during the operation, such as a pair of forceps. The arm unit 120 may be equipped with any other surgical instrument in which it is desired for its position to be fixed with high accuracy during the operation. By moving and fixing the surgical instrument using the medical observation apparatus 10 according to the embodiment, the position of the surgical instrument can be controlled with higher accuracy, and the working efficiency during the operation can be improved more.

Additionally, the present technology may also be configured as below.

(1) A medical observation apparatus including:

a holding unit including
a transmission mechanism including
   a plurality of joint units placed in positions different from each other and configured to rotate in conjunction with each other around a rotation axis in the same direction,

wherein brakes are placed for at least two joint units of the plurality of joint units.

(2) The medical observation apparatus according to (1), wherein

the brakes have fixing forces different from each other.

(3) The medical observation apparatus according to (1) or (2), wherein

the brake provided on a tip side of the holding unit out of the brakes has a smaller fixing force than the brake provided on a root side of the holding unit.

(4) The medical observation apparatus according to any one of (1) to (3), wherein

a tip of the holding unit is provided with a microscope unit for observing a surgical site with magnification.

(5) The medical observation apparatus according to (4), wherein

the microscope unit includes an imaging unit configured to output image information of the surgical site.

(6) The medical observation apparatus according to (5), wherein

the imaging unit is provided with an electronic zoom function.

(7) The medical observation apparatus according to any one of (1) to (6), wherein

the transmission mechanism is a parallelogram link mechanism including four arms arranged in a configuration of a parallelogram, and joint units provided individually in positions corresponding to vertices of the parallelogram.

(8) The medical observation apparatus according to (7), wherein

the brakes are placed for joint units provided at both ends of the arm with a relatively strong rigidity out of the four arms included in the parallelogram link mechanism.

(9) The medical observation apparatus according to any one of (1) to (8), wherein

a root side of the holding unit is provided with a counterweight.

(10) A medical video microscope apparatus including:

a holding unit including
a transmission mechanism including
   a plurality of joint units placed in positions different from each other and configured to rotate in conjunction with each other around a rotation axis in the same direction; and

a microscope unit provided at a tip of the holding unit and including an imaging unit configured to output image information of a surgical site imaged,

wherein brakes are placed for at least two joint units of the plurality of joint units in the holding unit.

(11) A medical video microscope system including:

a video microscope apparatus configured to acquire image information of a surgical site; and

a display device configured to display an image based on the image information,

wherein the video microscope apparatus includes

a holding unit including
a transmission mechanism including
   a plurality of joint units placed in positions different from each other and configured to rotate in conjunction with each other around a rotation axis in the same direction, and

a microscope unit provided at a tip of the holding unit and including an imaging unit configured to output the image information imaged, and

wherein brakes are placed for at least two joint units of the plurality of joint units in the holding unit of the video microscope apparatus.

What is claimed is:

1. A medical observation apparatus comprising:
a holding unit including
a transmission mechanism including
a plurality of joint units placed in positions different from each other and configured to rotate in conjunction with each other around a rotation axis in the same direction,
wherein brakes are placed for at least two joint units of the plurality of joint units.

2. The medical observation apparatus according to claim 1, wherein
the brakes have fixing forces different from each other.

3. The medical observation apparatus according to claim 1, wherein
the brake provided on a tip side of the holding unit out of the brakes has a smaller fixing force than the brake provided on a root side of the holding unit.

4. The medical observation apparatus according to claim 1, wherein
a tip of the holding unit is provided with a microscope unit for observing a surgical site with magnification.

5. The medical observation apparatus according to claim 4, wherein
the microscope unit includes an imaging unit configured to output image information of the surgical site.

6. The medical observation apparatus according to claim 5, wherein
the imaging unit is provided with an electronic zoom function.

7. The medical observation apparatus according to claim 1, wherein
the transmission mechanism is a parallelogram link mechanism including four arms arranged in a configuration of a parallelogram, and
joint units provided individually in positions corresponding to vertices of the parallelogram.

8. The medical observation apparatus according to claim 7, wherein
the brakes are placed for joint units provided at both ends of the arm with a relatively strong rigidity out of the four arms included in the parallelogram link mechanism.

9. The medical observation apparatus according to claim 1, wherein
a root side of the holding unit is provided with a counterweight.

10. A medical video microscope apparatus comprising:
a holding unit including
a transmission mechanism including
a plurality of joint units placed in positions different from each other and configured to rotate in conjunction with each other around a rotation axis in the same direction; and
a microscope unit provided at a tip of the holding unit and including an imaging unit configured to output image information of a surgical site imaged,
wherein brakes are placed for at least two joint units of the plurality of joint units in the holding unit.

11. A medical video microscope system comprising:
a video microscope apparatus configured to acquire image information of a surgical site; and
a display device configured to display an image based on the image information,
wherein the video microscope apparatus includes
a holding unit including
a transmission mechanism including
a plurality of joint units placed in positions different from each other and configured to rotate in conjunction with each other around a rotation axis in the same direction, and
a microscope unit provided at a tip of the holding unit and including an imaging unit configured to output the image information imaged, and
wherein brakes are placed for at least two joint units of the plurality of joint units in the holding unit of the video microscope apparatus.
